(19) United States
(12) Patent Application Publication
(10) Pub. No.: US 2023/0230690 A1
(43) Pub. Date: Jul. 20, 2023

(54) MEDICAL DEVICE MESSAGE CODING MANAGEMENT

(71) Applicant: ICU Medical, Inc., San Clemente, CA (US)

(72) Inventors: Rahul K R, Mesa, AZ (US); Mark C. Rohlwing, Mesa, AZ (US); Anandaraja S.; Bindu Malathi Prathapaneni, III; Satyendra Singh Jadaun, Kota (IN); Rosaiah Allam, Chennai (IN); Hrishikesh Anil Dandekar, Pune (IN)

(21) Appl. No.: 18/187,387

(22) Filed: Mar. 21, 2023

Related U.S. Application Data
(63) Continuation of application No. PCT/US2022/077705, filed on Oct. 6, 2022.

(30) Foreign Application Priority Data
Oct. 22, 2021 (IN) 202111048106

(51) Int. Cl. G16H 40/63; G16H 20/17

(52) U.S. Cl. CPC G16H 40/63; G16H 20/17

(57) ABSTRACT

Techniques for managing encoded communications for medical devices in a clinical environment are provided. Different versions of signal coding libraries are generated for different devices in a communication path. A first signal coding library may be generated using a first signal definition that includes a set of fields. A second signal coding library may be generated using a second signal definition that includes a subset of the fields of the first signal definition, and excludes one or more of the fields of the first signal definition. A message encoded using the first signal coding library may not be completely decodable using the second signal coding library. By selectively deploying the signal coding libraries to different systems, devices, and components in a clinical environment, access to information in message fields can be effectively managed.

### CROSS-REFERENCE TO RELATED APPLICATIONS

All applications for which a foreign or domestic priority claim is identified in the Application Data Sheet as filed with the present application are hereby incorporated by reference and made part of this specification.

### TECHNICAL FIELD

This disclosure relates to the field of medical device management, and particularly to systems and methods for communication with medical devices.

### BACKGROUND

Electronic medical devices often have processors and other computing components. Such medical devices may execute software and communicate with other computing systems via a network. Secure network communication may involve the establishing secure connections based on the identities of the devices participating in the communication, and encryption and decryption of data transmitted via the secure connections.

### SUMMARY

Various techniques for managing encoded communications for medical devices in a clinical environment are described herein. These techniques may include generating different versions of signal coding libraries for different devices in a communication path. A first signal coding library may be generated using a first signal definition that includes a set of fields. A second signal coding library may be generated using a second signal definition that includes a subset of the fields of the first signal definition, and excludes one or more of the fields of the first signal definition. A message encoded using the first signal coding library may not be completely decodable using the second signal coding library. Thus, the second signal coding library may be considered a limited signal coding library, and the first signal coding library may be considered an expanded or full signal coding library. By selectively deploying the limited and expanded signal coding libraries to different systems, devices, and components in a clinical environment, access to information in message fields can be effectively managed. For example, an intermediary such as a connectivity engine of a medical device can be provisioned with a limited signal coding library that provides access to only the portions of a message needed to manage and route messages, while shielding other fields from being accessed by the intermediary. The inaccessible data for the other fields may be passed through by the intermediary to other devices that may have an expanded signal coding library and may therefore be able to access the fields. These and other embodiments are described in greater detail below with reference to FIGS. 1-6. Although many of the examples are described in the context of medical devices, functions, and environments (including infusion pumps, medication dispensing functions, and hospital or clinical environments), the techniques described herein can be applied to other types of devices, functions, and environments.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an example medical device and computing system according to some embodiments.

FIG. 2 is a block diagram illustrating a message being encoded using an expanded signal coding library, and decoded using a limited signal coding library

FIG. 3 is a flow diagram of an illustrative routine to decode and encode a message using a limited signal coding library according to some embodiments.

FIG. 4 is a block diagram illustrating a message being encoded using a limited signal coding library, and decoded using another expanded signal coding library according to some embodiments.

FIG. 5 is a flow diagram of an illustrative routine to generate and distribute signal coding libraries according to some embodiments.

FIG. 6 is a block diagram illustrating data flows and interactions for generation and distribution of expanded and limited signal coding libraries according to some embodiments.

### DETAILED DESCRIPTION OF ILLUSTRATIVE EMBODIMENTS

The present disclosure is directed to managing the generation and processing of messages using various coding libraries to protect certain information from being altered or otherwise accessed by intermediaries in a clinical environment.

Some existing methods of message communication involve sending messages from a source to a target via an intermediary. For example, a message source may generate a message that includes instructions (e.g., auto-programming messages) and/or data to be acted upon by a message target. The message may be passed via one or more intermediaries for a variety of reasons, such as to modify or augment the message, to shield the target from direct communication, etc. Intermediaries may serve as a kind of a proxy or vanguard for security shielding the message target. Security mechanisms like encryption, authentication, tunneling, and the like may be implemented between the intermediary and the message source. Communication between the intermediary and message target may therefore not be required to go through these security mechanisms again. However, by passing the message to the target via an intermediary, the contents of the message may be exposed unnecessarily, which can be problematic if the intermediary has programming defects or is compromised.

Some aspects of the present disclosure address the issues noted above, among others, through the use of different coding libraries for encoding/decoding messages. In some embodiments, a message—also referred to herein as a signal-may be encoded such that different portions of the message are accessible separately from other portions of the message. A signal coding library may include a set of field definitions, and different fields may be assigned different values when a message is being generated. To transmit the message, it may be encoded using the signal coding library into an encoded form (e.g., a binary form) suitable for transmission. A recipient of the message may use a copy or version of the signal coding library to decode the message and access the values assigned to the various fields. If the recipient does not have a version of the signal coding library with the definition of a particular field, then the recipient will be unable to decode the portion that corresponds to that particular field, and will therefore be unable to modify or otherwise access the field, even inadvertently such as through programming defects. Such a limited signal coding library may be purposefully deployed to devices that serve as intermediaries. By providing limited signal coding libraries to intermediaries, the intermediaries can be prevented from modifying or even accessing particular portions of messages. However, the intermediaries can still be permitted to access and modify other portions of the messages. Moreover, the message communication protocol may be implemented such that the intermediary passes through, to a message target, those portions of the message that are not defined in the limited signal coding library used by the intermediary. If the target has a version of the signal coding library that defines fields in addition to those defined by the library used by the intermediary, then the target can decode the portions of the message passed through the intermediary and access the fields. In this way, a message source can communicate information to a message target via an intermediary without the intermediary being able to access some information, while still being able to access other information.

In some embodiments, individual fields or other portions of a message may be defined such that to the intermediary they are “read-only.” For example, a signal coding library may define one or more fields and corresponding access properties, including “optional,” “required,” “read-only,” or the like. When it is desirable to prevent access by the intermediary to a field altogether, the field can be left out of the limited signal coding library that is generated for the intermediary. When it is desirable to have the intermediary access certain fields for validation but not permit modification, the fields can be defined using the “read-only” property in the limited signal coding library generated for the intermediary. As with fields that are left out of the limited signal coding library but included in the expanded signal coding library, the expanded coding library may apply a property of “required” or “optional” and permit modification of values in fields that have the “read-only” property in the limited coding library.

Additional aspects of the present disclosure relate to generating and distributing different versions of signal coding libraries to different devices in a communication network. Some of the devices may be medical devices, such as infusion pumps, configured with program execution capabilities and network access. The medical devices may be configured to communicate with network computing devices for a variety of reasons, such as to send/receive data, to be remotely programmed to perform various medical procedures (administration of medication), etc. In some embodiments, a medical device may be provisioned with multiple distinct processors to segregate the functionality of the medical device. For example, a first processor (e.g., a user interface controller or “UIC”) may control the user interface, medication administration, and/or various other features of the medical device. A second processor (e.g., a communication engine or “CE”) may control network communications with other devices. Any communication to the medical device, even if targeted at the UIC, may be initially received by the CE. Thus, the CE may serve as an intermediary for all network communications to and/or from the medical device. Because of this, the CE is more exposed than the UIC to being compromised. In order to limit access to certain portions of messages (e.g., infusion program commands, medication administration data, etc.), the CE can be provided with a limited signal coding library. Because the CE has only a limited signal coding library, the CE will only be able to modify or otherwise access a limited set of fields of messages received by the medical device (e.g., excluding fields for infusion program commands, medication administration data, etc.). In contrast, the UIC can be provided with an expanded signal coding library. Thus, the UIC can access additional fields of a message that are merely passed through the CE (e.g., fields for infusion program commands, medication administration data, etc.).

In some embodiments, a message may include a hidden hash of the fields or other parts of a message that are to be inaccessible without the expanded signal coding library. When the target of a message (e.g., a UIC) receives a message via an intermediary (e.g., the CE), the target can use the hash to verify the contents of the fields that were supposed to be inaccessible to the intermediary (e.g., by hashing the contents of the fields and comparing to the hidden hash). In this way, even if the intermediary tampers—whether purposefully or inadvertently—with fields that are supposed to be inaccessible to the intermediary, the target can detect the tampering. In some embodiments, if the intermediary tampers with the fields that are supposed to be inaccessible to the intermediary a threshold number of times (e.g., 1 time, 3 times etc.), the target can lower a degree of trust in the intermediary, notify another computing system, or the like.

Various aspects of the disclosure will now be described with regard to certain examples and embodiments, which are intended to illustrate but not limit the disclosure. Although aspects of some embodiments described in the disclosure will focus, for the purpose of illustration, on particular examples of medical devices, message fields, encoding algorithms, coding libraries, and the like, the examples are illustrative only and are not intended to be limiting. In some embodiments, the systems and methods described herein may be applied to additional or alternative medical devices, message fields, encoding algorithms, coding libraries, etc.

### Overview of Example Network Environment

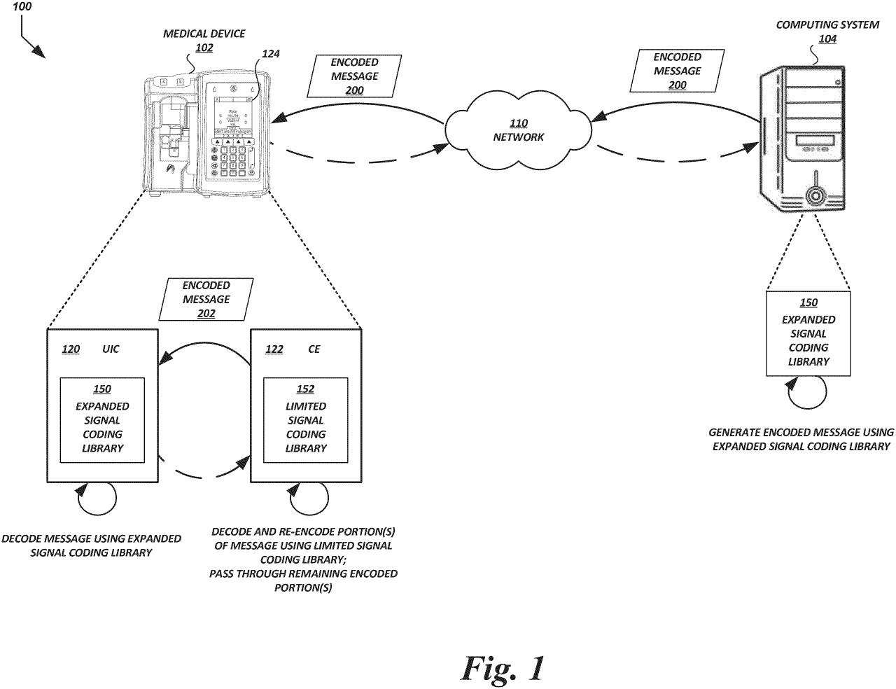

FIG. 1 shows a network environment 100 in which aspects of the present disclosure may be implemented for communicating using encoded messages and different versions of coding libraries. Devices of the network environment 100 may communicate with each other via one or more wired and/or wireless communication networks 110 such as local area networks (“LANs”), virtual local area networks (“VLANs”), wide area networks (“WANs”), etc. The network environment 100 may include any number of medical devices 102 and computing systems 104.

A medical device may 102 may be any electronic medical device, or medical device with an electronic component, configured to communicate with other devices over a communication network. In some embodiments, a medical device 102 may be an infusion pump.

The medical device 102 may include various subsystems and/or other components. In some embodiments, as shown, the medical device 102 may include a user interface controller 120 (“UIC”) to control aspects of a user interface of the medical device 102, such as a display screen 124. The UIC 120 may be implemented using computer-readable memory and a computer processor, such as a system on a chip (“SOC”). The medical device 102 may also include a connectivity engine 122 (“CE”) to manage network communications to and/or from the medical device, to provide network security for the medical device 102 (e.g., a firewall), and the like. The CE 122 may be implemented using computer-readable memory and a computer processor, such as an SOC. In some embodiments, the CE 122 may be implemented using a physically separate computer processor that than the computer processor on which the UIC 120 is implemented. In this way, the CE 122 can serve as an intermediary between the UIC 120 and other systems and devices of the network environment 100. If the CE 122 is compromised, its separation from the UIC 120 may provide an additional layer of protection to the UIC 120 and other components of the medical device 102.

In some embodiments, the medical device 102 may include additional and/or alternative components not shown. For example, if the medical device 102 is an infusion pump, the medical device 102 may include: a motor to administer medication from a medication container (e.g., a vial) through a tube to a patient; a motor controller to control the operation of the motor; a data store to store data and/or instructions used in operation of the medical device 102; various other components; or some combination thereof.

A computing system 104 may be any electronic device configured to electronically communicate with other devices over the network 110. In some embodiments, the computing system 104 may be a desktop computer, server computer, network appliance, or the like. The computing system 104 may send data and/or instructions to the medical device 102 for use in performing medical procedures, such as the administration of medication. In some embodiments, the computing system 104 may also or alternatively include one or more clinical IT systems. For example, the computing system 104 may be used to implement a hospital information system (“HIS”) designed to manage medical, administrative, financial, and/or legal issues of a medical facility in which the medical device 102 is used. The HIS can include one or more electronic medical record (“EMR”) or electronic health record (“EHR”) systems.

The example devices and systems of the network environment shown in FIG. 1 and described herein are illustrative only, and are not intended to be limiting, required, or exhaustive. In some embodiments, a network environment 100 may include additional, alternative, and/or fewer devices and/or systems. For example, although only one instance of a medical device 102 and computing system 104 are shown in FIG. 1, in practice any number or combination of devices and systems may be included in a network environment 100. A single network environment 100 may have dozens, hundreds, or more individual medical devices 102, and the medical devices 102 may be the same as or different than each other.

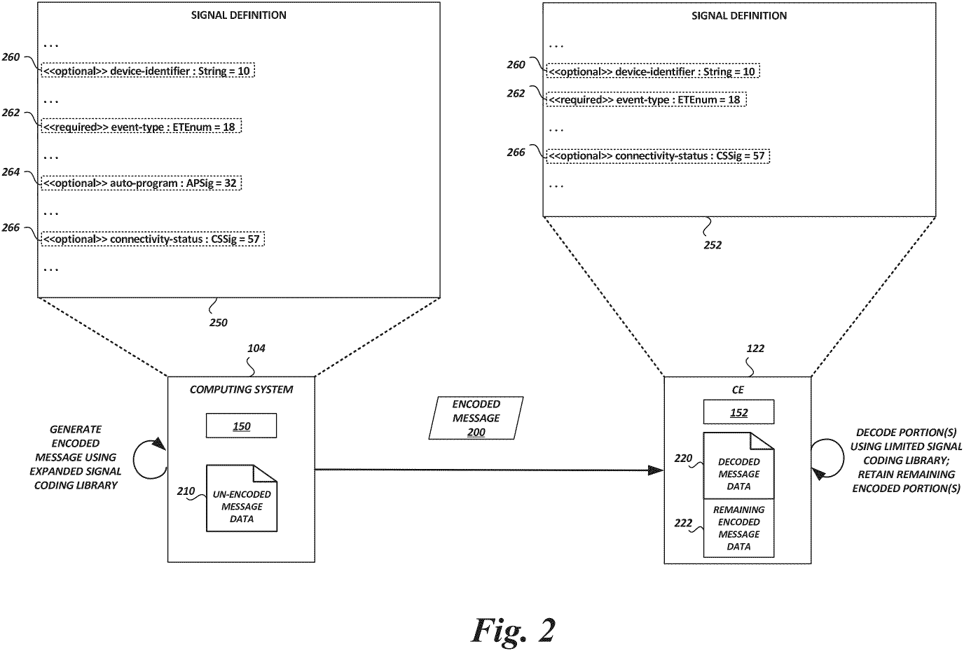

With reference to the example shown in FIG. 1, the computing system 104 may send an encoded message 200 to the medical device 102 via network 110. For example, the encoded message 200 may be an auto-programming message with information to program the medical device 102 to execute a process, such as to administer medication. The encoded message 200 may include various other information, such as timestamp information, type information, header information, and the like. The computing system 104 may generate an initial message object or structure in memory using a particular format that includes various fields and corresponding values for the fields. In order to transmit the message formed in memory, the computing system 104 may encode the message into a form that is suitable for transmission (e.g., binary form). The encoded message 200 may be generated using a signal coding library 150 that specifies how individual fields of the message 200 are to be encoded such that the resulting encoded message 200 may be transmitted to another device where it can be decoded and the values of the fields of the message 200 accessed. An example of using a signal coding library to generate an encoded message 200 is shown in FIG. 2 and described in greater detail below.

The encoded message 200 may be targeted to a component of the medical device 102, such as the UIC 120 which can use the encoded message 200 to perform various operations. As shown, the encoded message 200 may initially be received by the CE 122 of the medical device 102, which acts as an intermediary for communications to and/or from the UIC 120 and other components of the medical device 102. Although the CE 122 may be an intermediary rather than the intended target of all data included in the encoded message 200, the CE 122 may nevertheless be required or permitted to access various fields of the encoded message 200. For example, the CE 122 may access one or more fields to determine the type of the encoded message 200 for routing to the proper component of the medical device 102. As another example, the CE 122 may access a field to determine whether the CE 122 is to execute any operations, such as initiating retrieval of a file or other data to be used by another component of the medical device 102. As a further example, the CE 122 may access a field to modify information, such as a connectivity status, that another component of the medical device 102 may use. To access the fields, the CE 122 may use a signal coding library 152 to decode data from encode message 200 into a format that is accessible to the CE 122.

It may be desirable to prevent the CE 122 from accessing other fields of the encoded message 200. By preventing the CE 122 from accessing certain fields of the encoded message 200, those fields may remain uncompromised even if the CE 122 is compromised. For example, the CE 122 may be prevented from accessing auto-programming information. Thus, if the CE 122 is compromised, the auto-programming information that is sent in the encoded message 200 to the UIC 120 via the CE 122 may remain uncompromised.

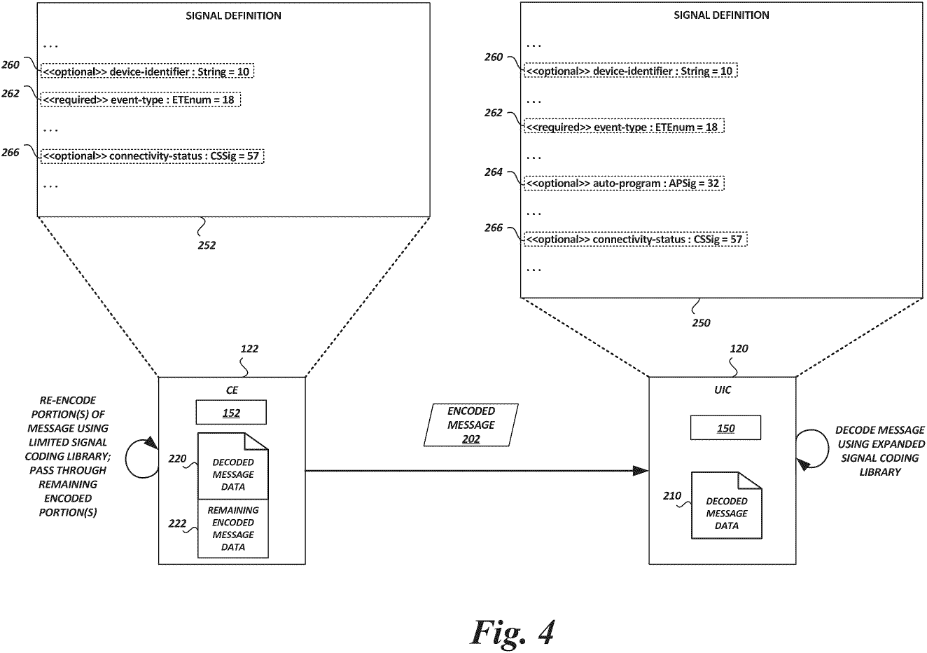

In order to prevent the CE 122 from accessing certain fields of the encoded message 200, the CE 122 may be provisioned with a different signal coding library than that used by the computing system 104. The signal coding library used by the CE 122 may be a limited signal coding library 152. In contrast to the expanded signal coding library 150 used by the computing system 104, the limited signal coding library 152 may not include definitions needed to decode certain fields from the message 200. For example, the limited signal coding library 152 may not include data needed to access any fields of which it may be desirable to prevent the CE 122 from accessing (e.g., auto-programming fields, procedure status fields, etc.). Advantageously, even though the CE 122 is unable to decode the fields for which it does not have a definition in the limited signal coding library 152, the CE 122 can retain the encoded data as retained encoded data, and pass it through to the UIC 120 as part of a second encoded message 202 that is sent from the CE 122 to the UIC 120. An example of generating such a second encoded message 202 including pass-through encoded data is shown in FIG. 4 and described in greater detail below.

Although the example shown in FIG. 1 relates to the CE 122 serving as an intermediary for, and passing messages to, the UIC 120, the example is illustrative only and is not intended to be limiting. In some embodiments, the CE 122 may also or alternatively serve as an intermediary for messages originating from the UIC 120 and targeted at other devices or systems such as computing system 104, as indicated by the dashed arrows. In some embodiments, the CE 122 may also or alternatively serve as an intermediary for communications to and/or from other components of the medical device 102, such as a pump motor controller, other controllers, other processors, or the like.

### Message Encoding and Selective Decoding Using Signal Definitions

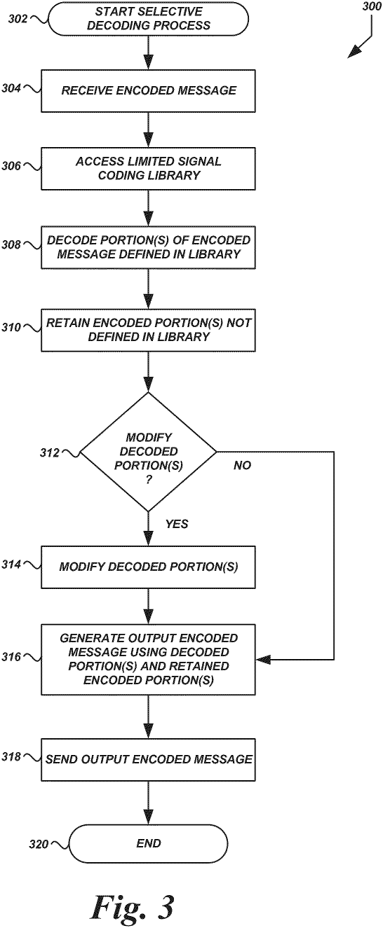

FIG. 2 illustrates an example original encoded message 200 that is encoded using an expanded signal coding library 150, and selectively decoded by an intermediary using a limited signal coding library 152. FIG. 3 is a flow diagram of an illustrative routine for decoding an encoded message 200 using a limited signal coding library 152, and generating a second encoded message 202—including pass-through encoded information—using the limited signal coding library 152. FIG. 4 illustrates receipt and subsequent decoding of the second encoded message 202 using an expanded signal coding library 150, which may be the same as or different from the expanded signal coding library used to encode the original encoded message 200. Advantageously, use of the expanded signal coding library 150 to decode the second encoded message 202 can permit access to information that was inaccessible to the intermediary due to the intermediary’s use of a limited signal coding library 152.

The original encoded message 200 and second encoded message 202 may include any number of fields, depending on the data to be sent and the requirements specified in the expanded signal coding library 150. FIG. 2 shows a graphical representation of a signal definition 250, including the fields that an expanded signal coding library 150 may be configured to encode and/or decode in a message. In the illustrated embodiment, some of the fields, such as field 262, are indicated as “«required»” and must be present in any message that is to be encoded/decoded using the expanded signal coding library 150 that is generated using this signal definition 250. Other fields, such as fields 260, 264, and 266, are indicated as “«optional»” and may or may not be present in any given message that is to be encoded/decoded using the expanded signal coding library 150.

Individual fields may be identified using labels and/or identification numbers. In addition, individual fields may be defined in terms of the type and/or structure of data to be included in the fields. For example, a field may be defined for data of a particular data type, such as a string, integer, floating point number, etc. As another example, a field may be defined for an enumeration, such as a set of possible numeric values mapped in a one-to-one manner to a set of possible alphanumeric tokens. As a further example, a field may be defined for a separate data structure, such as another signal definition that may itself include any number fields, field data types or structures, etc. In this way, signal data structures may be nested multiple levels deep, re-used across different top-level signals, etc.

In the illustrated embodiment, field 260 is an optional field uniquely identified using the identification number 10 and label “device-identifier.” Field 260 is defined as a string data type, and may therefore be assigned a string of characters. For example, field 260 may be used to identify the device that is the source or target of the message. Field 260 may be assigned a unique identifier of the device, such as a serial number.

In some embodiments, base data types such as strings may be used to signal data in a complex multi-component manner. For example, field 260 may be a fixed-length string in which the first x characters are used to signal the manufacturer of the device, the next y characters are used to signal the model of the device, and the last z characters are used to signal the unique identifier of the device (where x, y, and z are integers).

In the illustrated embodiment, field 262 is a required field uniquely identified using the identification number 18 and the label “event-type.” Field 262 is defined as an “ETEnum” enumeration field, and may therefore be assigned a value that corresponds to one of the enumerated values indicating which event caused the generation of the current message. For example, the values may include a 1 for an alarm event, a 2 for an auto-program event, a 5 for a software update event, etc.

In the illustrated embodiment, field 264 is an optional field uniquely identified using the identification number 32 and the label “auto-program.” Field 264 is defined as an “APSig” signal field, and may therefore be assigned values according to another signal definition. For example, the “APSig” signal definition may be structured with any number of fields, each field having an identifier and data type or structure, etc. Fields of the “APSig” signal definition may be used to provide medical device auto-programming information, such as data and/or instructions for performing a procedure (e.g., administration of medication). In some embodiments, signal fields used in one signal definition may include additional signal fields, resulting in signal definitions (also referred to as field definitions) nested in multiple levels.

In the illustrated embodiment, field 266 is an optional field uniquely identified using the identification number 57 and the label “connectivity-status.” Field 266 is defined as an “CSSig” signal field, and may therefore be assigned values according to another signal definition, in a manner similar to that described above with respect to field 264.

The example fields, data types, and structures shown in the figures and described herein are illustrative only, and are not intended to be limiting, required, or exhaustive. In some embodiments, additional and/or alternative fields, data types, and structures may be used.

A device may use a signal coding library to encode a message to be sent to another device. Encoding a message may involve converting in-memory data into an encoded form (e.g., a binary form) suitable for transmission. The signal coding library can be used to determine the manner in which individual fields of message data are to be converted into an encoded representation, and the manner in which the encoded representations are to be included in the encoded message such that they can be identified and decoded by the recipient.

With reference to an illustrative example, the computing system 104 can obtain un-encoded message data 210 representing an auto-programming message to be transmitted to a target, such as a medical device 102. The un-encoded message data 210 can include data representing values for fields 260, 262, 264, and 266, among others. The computing system 104 can use the expanded signal coding library 150 to generate encoded message 200 from the un-encoded message data 210. For example, the expanded signal coding library 150 can be used to encode the string value for the “deviceId” field into encoded form. The expanded signal coding library 150 can be further used to include the encoded data in the encoded message 200 using the corresponding unique identifier (2 in this example) of the “deviceId” field such that a recipient can identify the data and decode it into string form. A similar process may be used for each other field to be included in the encoded message 200. The computing system 104 can send the encoded message 200 to a recipient such as a medical device 102, a particular component thereof such as a UIC 120, or through an intermediary such as a CE 122.

The CE 122 may have its own signal coding library to decode messages and encode messages. However, as shown in FIG. 2, the CE 122 may have a limited signal coding library 152 rather the same expanded signal coding library 150 of the computing system 104. The limited signal coding library 152 may be limited in the sense that it does not define all of the fields that are defined in the expanded signal coding library 152. For example, FIG. 2 shows a graphical representation of a signal definition 252 from which the limited signal coded library 152 may have been generated. The signal definition 252 includes a subset of fields of signal definition 250 (e.g., fields 260, 262, and 266, among others), but does not include a definition of other fields (e.g., field 264, among others). Therefore, the CE 122 can use the limited signal coding library 152 to decode and access the information in fields 260, 262, and 266, but not field 264.

FIG. 3 is a flow diagram of an illustrative routine 300 that may be used by a message recipient with a limited signal coding library (such as CE 122 with limited signal coding library 152) to process messages encoded with an expanded signal coding library. The routine 300 will be described with reference to the data flows and signal coding libraries shown in FIGS. 2 and 4. The routine 300 begins at block 302.

At block 304, a message recipient may receive an encoded message. In the example shown in FIG. 2, a CE 122 may receive encoded message 200 from computing system 104. Continuing with the example introduced above, the encoded message 200 may be an auto-program message providing information to an infusion pump regarding an administration of medication. The encoded message 200 may include various fields defined by the expanded signal coding library 150, such as fields 260, 262, 264, and 266, among others. The encoded message 200 may exclude other optional fields defined by the expanded signal coding library 150.

At block 306, the message recipient can access a signal coding library to decode the encoded message 200. In the example shown in FIG. 2, the CE 122 has a limited signal coding library 152.

At block 308, the message recipient can decode the portion or portions of the encoded message 200 for which the available signal coding library is configured to decode. In the example shown in FIG. 2, the CE 122 may use limited signal coding library 152 to generate decoded message data 220 by decoding fields 260, 262, and 266 because limited signal coding library 152 has the necessary data and/or instructions to identify and decode the encoded data that corresponds to fields 260, 262, and 266.

In some embodiments, decoding a message or a particular field thereof using a signal coding library may involve analyzing the message for a field identifier. For example, as shown in FIG. 2, field 260 is identified using identification number 2. The signal coding library can be used to analyze the encoded message data for data identifying the location or portion of data for the field identified using identification number 2. In addition to the identification number, the signal coding library can be used to interpret, de-serialize, or otherwise decode the encoded data for the field identified using identification number 2. In this example the field is defined as a string, and the signal coding library can be used to decode the data into the string value represented by the data. Notably, without a signal coding library configured with the definition of the field 260 identified by identification number 5 and data type string, the data for field 260 would be unable to be decoded.

In some embodiments, a label for a field may be modified while maintaining a consistent field identifier. One signal coding library may use one label for a particular field identified by a particular field identifier, while another coding library may use a different label for a different field identified by the same field identifier. In such case, the field encoded by one signal encoding library will still be able to be decoded using the other signal coding library based on the field identifier. For example, field 260, identified using identification number 2, may have the label “deviceID” when encoded using an expanded coding library, and may have the label “pumpID” when encoded using the limited coding library. Thus, different users of different signal coding libraries may locally define a field differently without being tied to how other signal coding libraries label the fields.

At block 310, the message recipient can retain any remaining encoded portions of encoded message 200 that the available signal coding library is not configured to decode. In the example shown in FIG. 2, the limited signal coding library 152 does not include a definition for field 264, and thus the limited signal coding library 152 is not able to be used to decode the encoded data that corresponds to field 264. The portion of the encoded message 200 that is unable to be decoded using the limited signal coding library 152, including the data for field 264, can be retained in encoded form as remaining encoded message data 222.

At block 312, the message recipient can determine whether any portions of the message are to be modified. If so, the routine 300 may proceed to block 314. Otherwise, the routine may proceed to block 316. In some embodiments, individual fields or other portions of a message may be defined such that they are “read-only.” For example, a signal coding library may define one or more fields and corresponding access properties, including “optional,” “required,” “read-only,” or the like. When it is desirable to prohibit an entity from accessing certain fields, the fields can be defined using the “read-only” property in the signal coding library (e.g., the limited signal coding library generated for the intermediary). Such fields may not be permitted to be modified when reading and encoding a message to be sent to a subsequent recipient using the signal coding library.

In the example shown in FIG. 4, the CE 122 can determine that the value for field 266 is to be modified (or provided, if no value is currently assigned to field 266). For example, the CE 122 may use field 266 to communicate current connectivity status information to the UIC 120. The CE 122 may determine the current connectivity status, and update the value (or values) of the field 266 accordingly at block 314.

At block 316, the message recipient can generate an output encoded message using the decoded portions and the retained encoded portions that the recipient was unable to decode. In the example shown in FIG. 4, the CE 122 can use the limited signal coding library 152 to encode the decoded message data 220 for fields 260, 262, and 266. The encoded data for those fields, with the remaining encoded message data 222, may be used to generate an output encoded message 202.

At block 318, the message recipient can send the output encoded message to the target recipient or otherwise to the next recipient. In the example shown in FIG. 4, the CE 122 may send the output encoded message 202 to the UIC 120. Advantageously, the UIC 120 has an expanded signal coding library 150 that is configured to decode all fields of encoded message 202, including those that correspond to the portions of the encoded message 200 (e.g., the remaining encoded message data 222) that the CE 122 was unable to decode using the limited coding library 152.

The routine 300 may terminate at block 320.

### Selective Signal Coding Library Generation and Distribution

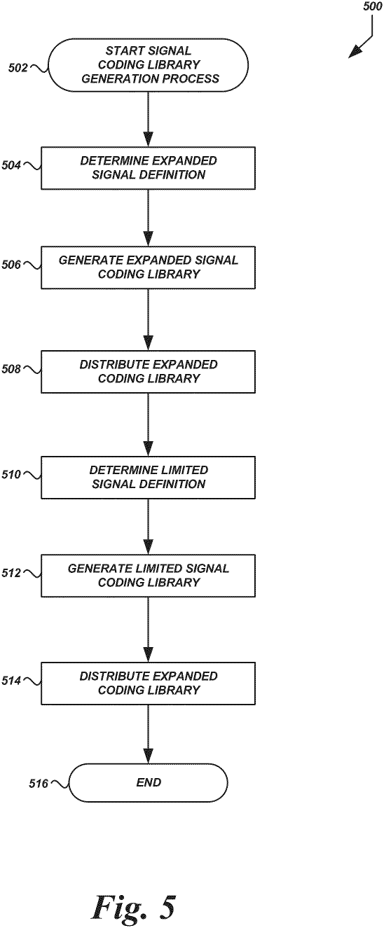

FIG. 5 is a flow chart of an illustrative routine 500 for generating and distributing different signal coding libraries. Advantageously, the different signal coding libraries may include a library configured to encode and decode a set of message fields, and another library configured to encode and decode a subset of those message fields such that second library is not configured to encode and decode all fields that the first library is configured to encode and decode. Thus, the first library may be considered to be an expanded or full signal coding library and the second library may be considered to be a limited signal coding library. In this way, messages may be passed between systems and devices, and certain systems and devices may be prevented from accessing certain fields of the messages while still being able to access other fields of the messages.

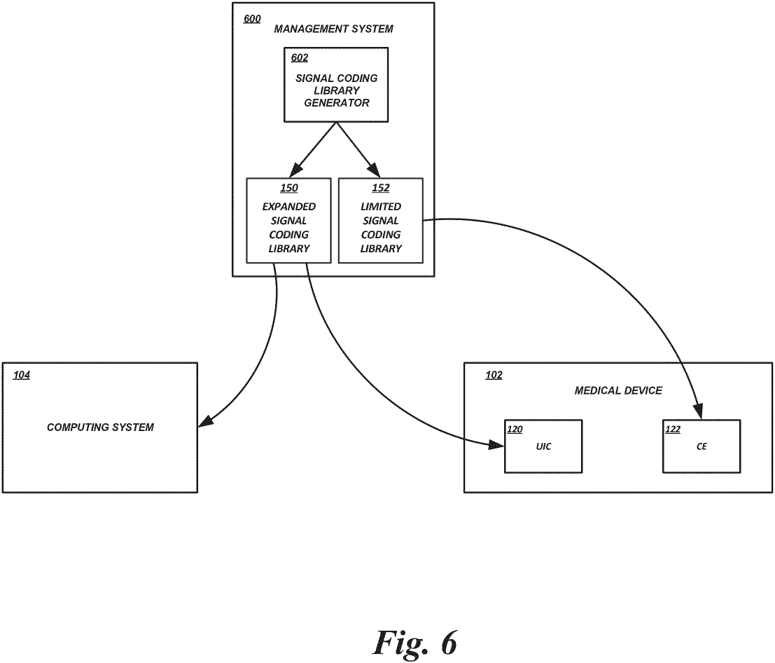

The routine 500 begins at block 502. The routine 500 may begin in response to an event, such as in response to an interactive command to generate and distribute different signal coding libraries for communicating messages that include data from a set of fields. The routine 500 will be described with further reference to the illustrative data flows and interactions shown in FIG. 6.

FIG. 6 illustrates a management system 600 that includes a signal coding library generator 602. The management system 600, also referred to as a management device, may be a computing device or set of computing devices, such as desktop computing devices, server computing devices, midrange computing devices, a set of cloud-based computing resources, or the like. The signal coding library generator 602 may implemented as a dedicated hardware component, or as a combination of hardware and software.

At block 504, the signal coding library generator 602 can determine a set of message fields for which a first signal coding library is to be configured to encode and decode data. In the example shown in FIG. 6, the first signal coding library may be an expanded signal coding library 150 that is configured to encode and decode all possible fields that can be included in an encoded message, such as the fields from signal definition 250 shown in FIG. 2.

At block 506, the signal coding library generator 602 can generate the first signal coding library based on the set of message fields determined above. To generate the first signal coding library, the signal coding library generator 602 may generate executable code configured to encode each field into encoded form into a message, and decode the encoded data into a form usable by other devices and components.

At block 508, the management system 600 can distribute the first signal coding library to one or more devices. In the example shown in FIG. 6, the management system 600 may distribute the expanded signal coding library 150 to the computing system 104 and to the UIC 120. In some embodiments, the expanded signal coding library 150 may be provided directly to the UIC 120, such as when the medical device 102 is manufactured or being readied for deployment. In some embodiments, the expanded signal coding library 150 is sent to the medical device 102, where it is received by the CE 122 and forwarded to the UIC 120.

At block 510, the signal coding library generator 602 can determine a subset of message fields for which a second signal coding library is to be configured to encode and decode data. In the example shown in FIG. 6, the second signal coding library may be a limited signal coding library 152 that is configured to encode and decode a subset of all possible fields that can be included in an encoded message, such as the fields from signal definition 252, which are a subset of the fields from signal definition 250.

At block 512, the signal coding library generator 602 can generate the second signal coding library based on the subset of message fields determined above. To generate the second signal coding library, the signal coding library generator 602 may generate executable code configured to encode each field into encoded form into a message, and decode encoded data into a form usable by other devices and components. Any data in an encoded message that is not defined or otherwise decodable using second signal coding library may be retained in encoded form to be included in a subsequent message encoded using the second signal coding library.

At block 514, the management system 600 can distribute the second signal coding library to one or more devices. In the example shown in FIG. 6, the management system 600 may distribute the limited signal coding library 152 to the CE 122. In some embodiments, the limited signal coding library 152 may be provided directly to the CE 122, such as when the medical device 102 is manufactured or being readied for deployment. In some embodiments, the limited signal coding library 152 is sent to the medical device 102, where it is received by the CE 122 and employed in use.

The routine 500 may terminate at block 516.

In some embodiments, the management system 600 can generate signal coding library updates to enable new features available to the medical device 102 and/or computing system 104. For example, the UIC 120 and computing system 104 may be updated with or otherwise capable of providing additional features that are not supported by the existing signal coding libraries. The management system 600 can create new expanded signal coding libraries 150 and provide them to the medical device 102 and computing system 104 to enable the features. At the medical device 102, the new expanded signal coding library 150 may be used by the UIC 120 in place of a prior expanded signal coding library 150 to decode and encode messages to facilitate the additional features. The CE 122, however, may not necessarily receive an updated limited signal coding library 152, but may nevertheless be capable of passing message data encoded using the new expanded signal coding library 150 between the computing system 104 and UIC 120, as described herein. In this way, the CE 122 may be shielded from any impact of such feature changes and corresponding changes to expanded signal coding libraries 150.

In some embodiments, signal coding libraries may be tagged or otherwise associated with version data. The version data may be used to implement a version tracking scheme to avoid incompatibility between various versions of limited and expanded signal libraries. For example, a message encoded using a particular version of a signal coding library—whether a limited or expanded signal coding library—may be tagged with or otherwise incorporate data indicating the version of the library used. Subsequently when the message is to be decoded—whether using a limited signal coding library or expanded signal coding library—the version data may be evaluated to ensure that the proper signal coding library version is used to decode the message.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a computer processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A computer processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A system for managing encoded communications for medical devices, the system comprising:
a management device; and
a medical device comprising a communication engine and a user interface controller;
wherein the management device is configured to:
generate an expanded signal coding library based on a first signal definition, wherein the first signal definition comprises a set of field definitions;
provide the expanded signal coding library to the user interface controller;
generate a limited signal coding library based on a second signal definition, wherein the second signal definition includes a first subset of the set of field definitions and excludes a second subset of the set of field definitions; and
provide the limited signal coding library to the communication engine;
wherein the communication engine is configured to:
receive a first encoded message;
decode a first portion of the first encoded message using the limited signal coding library to generate first decoded message data, wherein the first decoded message data comprises data representing at least a first field from the first subset of fields, the first field comprising timestamp data;
retain a second portion of the first encoded message as retained encoded message data, wherein the retained encoded message data comprises data representing at least a second field from the second subset of fields, the second field comprising auto-programming information to program the medical device to administer medication;
generate a second encoded message using the limited signal coding library, wherein the second encoded message comprises the retained encoded message data and encoded data representing the first field; and
wherein the user interface controller is configured to:
receive the second encoded message; and
decode the second encoded message using the expanded signal coding library to generate second decoded message data, wherein the second decoded message data comprises data representing the first field and the second field.

2. The system of claim 1, wherein the communication engine is further configured to modify a value associated with the first field, wherein the encoded data representing the first field in the second encoded message represents the value that has been modified.

3. The system of claim 1, wherein the first encoded message, received by the communication engine, is encoded using the expanded signal coding library.

4. The system of claim 1, wherein the user interface controller is further configured to:
generate a third encoded message using the expanded signal coding library; and
provide the third encoded message to the communication engine.

5. The system of claim 4, wherein the communication engine is further configured to:
receive the third encoded message;
decode a first portion of the third encoded message using the limited signal coding library to generate third decoded message data, wherein the third decoded message data comprises data representing at least the first field;
retain a second portion of the third encoded message as second retained encoded message data; and
generate a fourth encoded message using the limited signal coding library, wherein the fourth encoded message comprises encoded data representing the first field, and wherein the fourth encoded message comprises the second retained encoded message data.

6. An infusion pump comprising:
a first processor configured to:
receive a first encoded message;
decode a first portion of the first encoded message using a first signal coding library to generate first decoded message data representing at least a first field;
retain a second portion of the first encoded message as retained encoded message data comprising data representing at least a second field; and
generate a second encoded message using the first signal coding library, wherein the second encoded message comprises the retained encoded message data and encoded data representing the first field;
a second processor configured to:
receive the second encoded message; and
decode the second encoded message using a second signal coding library to generate second decoded message data representing the first field and the second field;
a display configured to display information based at least partly on the second decoded message data; and
a motor controller configured to cause medication to be administered from a medication container.

7. The infusion pump of claim 6, wherein the first field comprises data representing at least one of: message timestamp information, message type information, or message header information.

8. The infusion pump of claim 6, wherein the second field comprises data representing at least one of: auto-programming message information to program the infusion pump to administer medication, or procedure status information.

9. The infusion pump of claim 6, wherein the first processor is further configured to modify a value associated with the first field, wherein the encoded data representing the first field in the second encoded message represents the value that has been modified.

10. The infusion pump of claim 6, wherein the first encoded message, received by the first processor, is encoded using the second signal coding library.

11. The infusion pump of claim 6, wherein the second processor is further configured to:
generate a third encoded message using the second signal coding library, wherein the third encoded message comprises data representing at least a third field and a fourth field; and
provide the third encoded message to the first processor.

12. The infusion pump of claim 11, wherein the first processor is further configured to:
receive the third encoded message;
decode a first portion of the third encoded message using the first signal coding library to generate third decoded message data, wherein the third decoded message data comprises data representing at least the third field;
determine that a second portion of the third encoded message is unable to be decoded using the first signal coding library;
retain the second portion of the third encoded message as second retained encoded message data, wherein the second retained encoded message data comprises data representing at least the fourth field; and
generate a fourth encoded message using the first signal coding library, wherein the fourth encoded message comprises encoded data representing the first field, and wherein the fourth encoded message comprises the second retained encoded message data.

13. The infusion pump of claim 6, wherein the first signal coding library defines the first field as having an access property comprising one of: optional, required, or read-only.

14. The infusion pump of claim 13, wherein the first signal coding library defines a third field as having a different access property than the first field.

15. A computer-implemented method comprising:
as performed by a computing system comprising one or more processors configured to execute specific instructions,
obtaining a first signal definition comprising a plurality of field definitions;
generating a first signal coding library based on the first signal definition;
providing the first signal coding library to a first processor, wherein the first processor uses the first signal coding library to decode encoded messages comprising data associated with at least one of the plurality of field definitions;
obtaining a second signal definition comprising a first subset of the plurality of field definitions, wherein the second signal definition does not include a second subset of the plurality of field definitions;
generating a second signal coding library based on the second signal definition; and
providing the second signal coding library to a second processor, wherein the second processor uses the second signal coding library to decode encoded messages comprising data associated with at least one of the first subset of field definitions.

16. The computer-implemented method of claim 15, wherein providing the first signal coding library to the first processor comprises providing the first signal coding library to a medical device comprising the first processor and the second processor, wherein the second processor serves as an intermediary for communications to the first processor.

17. The computer-implemented method of claim 15, further comprising providing the first signal coding library to a computing device separate from the first processor, wherein the computing device uses the first signal coding library to encode encoded messages comprising data associated with at least one of the plurality of field definitions.

18. The computer-implemented method of claim 15, wherein generating the first signal coding library is based on data, from the first signal definition, representing at least one of: auto-programming message information to program the second processor to administer medication, or procedure status information.

19. The computer-implemented method of claim 15, wherein generating the second signal coding library is based on data, from the first signal definition, representing at least one of: message timestamp information, message type information, or message header information.

20. The computer-implemented method of claim 15, wherein generating the second signal coding library comprises associating individual fields of the plurality of field definitions as one of: optional, required, or read-only.
